(19) United States
(12) Patent Application Publication
(10) Pub. No.: US 2020/0217847 A1
(43) Pub. Date: Jul. 9, 2020

(54) Method a System for a Biomarker Breath Test Using Mass Abnormalities in Gaseous Ions with Imaging Correlates

(71) Applicant: Michael PHILLIPS, Fort Lee, NJ (US)

(72) Inventor: Michael PHILLIPS, Fort Lee, NJ (US)

(21) Appl. No.: 16/735,118

(22) Filed: Jan. 6, 2020

Related U.S. Application Data
(63) Continuation-in-part of application No. 15/177,695, filed on Jun. 9, 2016.
(60) Provisional application No. 62/174,256, filed on Jun. 11, 2015.

(51) Int. Cl. G01N 33/574; G06T 7/00

(52) U.S. Cl. CPC G01N 33/57423; G06T 7/0012; G06T 2207/30004; G06T 2207/10081; G01N 2800/7028

(57) ABSTRACT

The present invention provides a method for identifying biomarkers and generating an output indicative of lung cancer. The method for identifying biomarkers comprises the steps of collecting a breath sample from subjects known to have nodules on a LDCT and subjects known to be free of nodules on the LDCT; analyzing the collected breath samples to determine all mass ions in each of the collected breath samples using at least one time-resolved separation technique and at least one mass-resolved separation technique; identifying a subset of the determined mass ions in a processor as the biomarkers for detecting lung cancer.

### BACKGROUND OF THE INVENTION

Previous studies have reported volatile organic compounds (VOCs) in breath as apparent biomarkers of lung cancer. Since seeking breath biomarkers of lung cancer, researchers have employed a wide range of different tools including VOC separation methods using gas chromatography mass spectrometry (GC MS), non-separative detectors, such as electronic noses and chemosensors, analysis of expired breath condensate, measurement of breath temperature, and sniffer dogs. Analysis of breath VOCs with analytical instruments employing 2-dimensional GC has revealed a complex matrix of 2,000 different VOCs in a single sample. Data management tools for metabolomic analysis that were originally developed for genomics and proteomics have been used to manage the information. An increased risk of false discovery of biomarkers can arise when a multivariate model over-fits large number of candidate breath VOCs to a small number of test subjects, these VOCs could have been non-specific biomarkers of malignant as well as non-malignant lung diseases, these VOCs could have been non-specific biomarkers of malignant as well as non-malignant lung diseases.

Despite these concerns, breath biomarkers of lung cancer have been proposed as safe and cost-effective tools to help determine a person's risk of lung cancer. There is a clinical need for such a test because more people in the United States die from lung cancer than from any other type of cancer and early detection can save lives. The National Lung Screening Trial found that screening with low-dose chest CT reduced mortality from lung cancer by 20%. However, the comparatively low positive predictive value (PPV) of chest CT (2.4% to 5.2%) has raised concerns that screening for lung cancer might yield an overwhelming number of false-positive test results.

It has also been found that chest imaging can cause harm. The National Lung Screening Trial 1,2 also revealed two major deficiencies of low-dose computed tomography of chest (LDCT). The LDCT showed low yield only 7.3% of subjects were positive for lung cancer on biopsy, and poor specificity since the false-positive rate was 20.1%. The resulting harms were: over-investigation of false-positive results with potentially harmful tests, such as bronchoscopy, biopsy. Needless exposure to potentially harmful radiation: 92.7% of the irradiated population was cancer-free. LDCT has high costs plus higher costs of needless tests to over-investigate false-positive results. Pulmonary nodules seen on LDCT frequently elicit false-positive reports of malignancy. False-positive reports can lead to needless procedures such as bronchoscopy and lung biopsy that are invasive, costly, and potentially hazardous.

Radiologists have attempted to minimize false-positive LDCT results by stratifying cancer risk according to the radiographic appearance of a nodule. Pulmonary nodule features seen on LDCT that are most suggestive of malignancy include lesion size >11 mm and ground-glass appearance, while polygonal lesions are usually benign.

The problem with predictions of malignancy based on the appearance of a pulmonary nodule is that no single feature is both highly sensitive and highly specific for disease. Researchers have attempted to improve the sensitivity and specificity of LDCT by employing various combinations of the nodule features shown using naked eye assessment as well as computer-assisted algorithms and machine learning with artificial neural networks. LDCT has also been combined with ancillary imaging modalities such as magnetic resonance imaging (MRI) and positron emission tomography (PET) scanning. The combination of LDCT with MRI and positron emission tomography (PET) scanning has the shortcoming of entailing additional costs and radiation exposure.

It is desirable to provide new and improved methods of predicting nodules on a chest CT with improved accuracy and reduce the number of false-positive and false-negative test findings.

### SUMMARY OF THE INVENTION

The present invention provides a method and system for identifying a non-invasive biomarker in breath for detecting lung cancer. The disclosed system and method far exceeds the sensitivity and reliability of conventional LDCT and can be used for reducing the number of false-positive reports of malignant pulmonary nodules. In the present invention, the method determines a single breath biomarker that can be used to predict nodules on a chest CT that are read as consistent with lung cancer. The biomarker is referred to as mass abnormalities in gaseous ions with imaging correlates (MAGIIC).

The method for identifying a biomarker to predict nodules on a chest CT as indicating lung cancer comprises the steps of:

collecting a breath sample from subjects known to have nodules on a chest CT and subjects known to be free of nodules on a chest CT;

analyzing the collected breath samples to determine all mass ions in each of the collected breath samples using at least one time-resolved separation technique and at least one mass-resolved separation technique;

identifying a subset of the determined mass ions in a processor as the biomarkers for detecting the disease; and

combining the subset of the determined mass ions in a multivariate algorithm in the processor to generate a value of a discriminant function indicating the likelihood that nodules on a chest LDCT of the subject are consistent with lung cancer.

In one embodiment, the biomarker is determined in breath from a single volatile organic compound (VOC) after bombardment of the breath VOC with high energy electrons using a gas chromatography mass spectrometry (GC MS). Alternatively, the VOC can be analyzed with a surface acoustic wave (SAW) gas chromatography sensor (GC SAW) or flame ion detection (GC FID).

The biomarkers in breath can be oxidative stress biomarkers. In one embodiment, the biomarkers in breath are C4 and C5 alkanes or alkane derivatives.

In one embodiment, the method of the present invention is used for predicting the probable presence of lung cancer in a test subject using the method for identifying biomarkers of the present invention.

Another embodiment of the invention features a system for identifying a plurality of biomarkers for predicting lung cancer in a subject including an apparatus for collecting a breath sample from subjects known to have nodules on a chest CT and subjects known to be free of nodules on a chest CT. A mass spectrometer (MS) associated with a gas chromatograph (GC) apparatus analyzes the collected breath samples to determine all mass ions in each of the collected breath samples. A computer identifies a subset of the determined mass ions as the biomarkers for detecting lung cancer as the disease, the subset of the determined mass ions indicate the likelihood that nodules on a chest LDCT of the subject are consistent with lung cancer, and combines the subset of the determined mass ions in a multivariate algorithm to generate a discriminant function. The discriminant function indicates a value of the likelihood that the subject has lung cancer.

It was found that biomarkers determined with the method of the present invention accurately predicted lung cancer in a blinded replicated study

The invention will be more fully described by reference to the following drawings.

### BRIEF DESCRIPTION OF THE DRAWINGS

The invention will be more fully described by reference to the following drawings.

FIG. 1 is flow diagram of a method for identifying a biomarker and generating an output indicative of predicting lung cancer in accordance with the teachings of the present invention.

FIG. 2 is a flow diagram of steps which can be used for identifying a subset of mass ions which are statistically significant for detecting disease.

FIG. 3 is a flow diagram of steps which can be used for selecting the biomarker mass ions with at least greater than random diagnostic accuracy.

FIG. 4 is a schematic diagram a system for identifying a biomarker for indicating lung cancer and for detecting lung cancer from the probability of identifying nodules in a CT scan accordance with the teachings of the present invention.

FIG. 5 is a plot of predicted sensitivity and specificity in a training set of subjects for predicting nodules observed on a LDCT.

FIG. 6A a plot of predicted sensitivity and specificity in a test set of subjects for predicting nodules observed on a LDCT at laboratory A in a blinded phase.

FIG. 6B a plot of predicted sensitivity and specificity in a test set of subjects for predicting nodules observed on a LDCT at laboratory B in a blinded phase.

FIG. 7 is a plot of sensitivity of the biomarker of the present invention referred to as mass abnormalities in gaseous ions with imaging correlates (MAGIIC) of LDCT.

### DETAILED DESCRIPTION

Reference will now be made in greater detail to a preferred embodiment of the invention, an example of which is illustrated in the accompanying drawings. Wherever possible, the same reference numerals will be used throughout the drawings and the description to refer to the same or like parts.

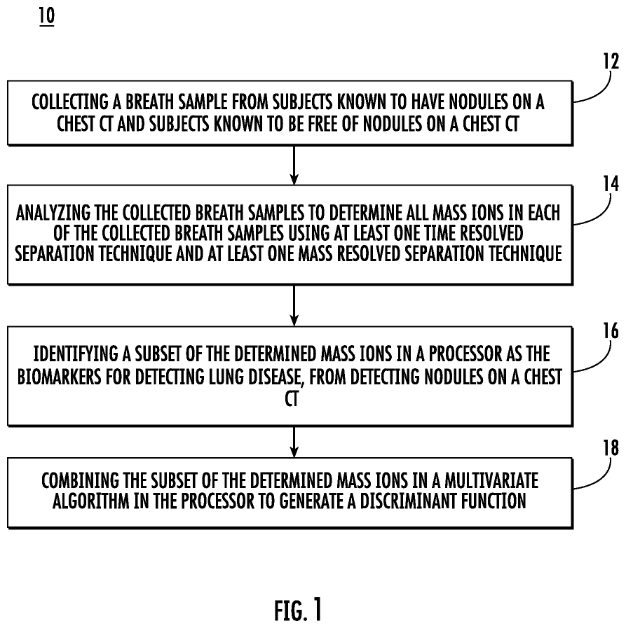

FIG. 1 is flow diagram of a method 10 for identifying a biomarker to predict nodules on a chest CT as indicating lung cancer. In block 12, breath samples are collected from subjects known to have nodules on a chest CT and subjects known to be free of nodules on a chest CT. In one embodiment, breath samples are collected from subjects known to have nodules on a LDCT. In block 14, the collected breath samples are analyzed to determine all mass ions in each of the collected breath samples using at least one time-resolved separation technique and at least one mass-resolved separation technique. In a preferred embodiment, the collected breath samples are analyzed with two parameters a mass to charge ration (m/z) and a chromatographic retention time. In one embodiment, the collected breath samples were analyzed with chromatography and mass spectrometry (GC MS). Data from the GC MS of chromatograms is processed in a computer processor to identify mass ions in the sample. Alternatively, the collected breath samples can be analyzed with a surface acoustic wave (SAW) gas chromatography sensor (GC SAW) or flame ion detection (GC FID).

In block 16, a subset of the determined mass ions are determined that correlated with the presence of structural abnormalities in LDCT that were read as consistent with nodules. In block 18, the subset of the determined mass ions is combined in a multivariate predictive algorithm to generate a value of a discriminant function (DF) indicating the likelihood that the subject has nodules on a LDCT.

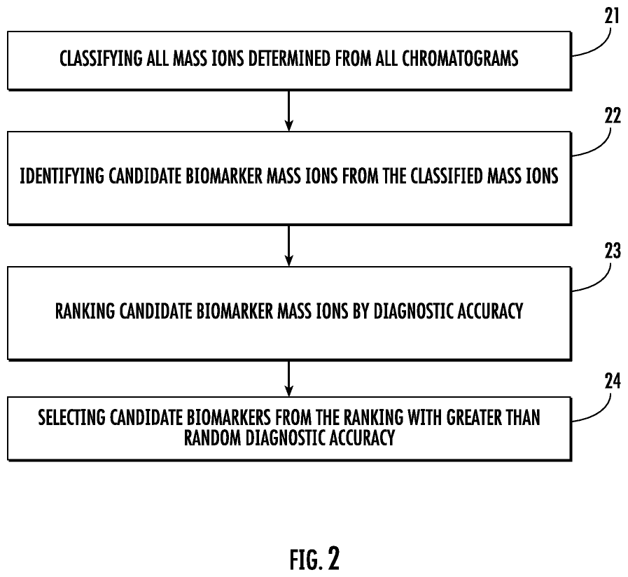

FIG. 2 is a flow diagram of steps which can be used for identifying the subset of mass ions which are statistically significant for detecting a likelihood that the subject has nodules on a LDCT. In block 21, all mass ions determined from all chromatograms of the breath samples are classified using intensities and retention times. Candidate biomarker mass ions from the classified mass ions are identified in block 22. The candidate biomarker mass ions are ranked by diagnostic accuracy for predicting the likelihood that the subject has nodules on a LDCT, in block 23. In block 24, the candidate biomarker mass ions with at least greater than random diagnostic accuracy are selected as the subset of mass ions which are statistically significant for predicting the likelihood that the subject has nodules on a LDCT.

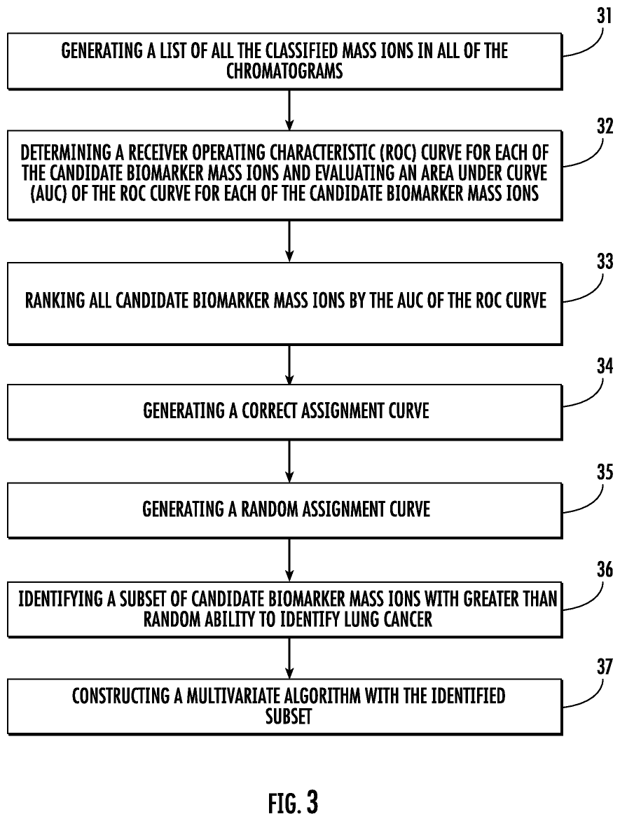

FIG. 3 is a flow diagram of steps which can be used for selecting the biomarker mass ions with at least greater than random diagnostic accuracy to be used in the multivariate predictive algorithm. In block 31, a list is generated of all the classified mass ions in all of the chromatograms. In block 32, the diagnostic accuracy is determined by determining a receiver operating characteristic (ROC) curve for each of the candidate biomarker mass ions and evaluating an area under curve (AUC) of the ROC curve for each of the candidate biomarker mass ions reflecting the sensitivity and specificity for predicting the likelihood that the subject has nodules on a LDCT. In block 33, all candidate biomarker mass ions are ranked by the AUC of the ROC curve. The ranking can be from highest to lowest.

Blocks 34, 35 and 36 describe steps using multiple Monte Carlo simulations to identify a set of mass ion biomarkers predicting the likelihood that the subject has nodules on a LDCT with greater than random accuracy. In block 34, a correct assignment curve is constructed with data of the AUC of the ROC curves for all candidate biomarker mass ions. In one embodiment, block 34 can be performed by assigning all data of the AUC of the ROC curves to a series of bins with incremental values. For example, the bins can be assigned values of 0.50 to 0.51, 0.51 to 0.52 and so forth up to 0.99 to 1.0. The correct assignment curve is generated as a plot of the number of mass ions in a bin on the y-axis versus the AUC value of a bin on the x-axis

In block 36, the subset of candidate biomarker mass ions with greater than random ability to identify nodules on a LDCT for identifying lung cancer are identified using a correct assignment curve and a random assignment curve

In block 37, the multi-variate predictive algorithm is constructed using the candidate biomarker mass ions from the correct assignment curve that were identified as having greater than random ability to identify predicting nodules on a LDCT. A list is generated of all candidate biomarker mass ions in the correct assignment curve that were identified as having greater than random ability to identify the disease. Each of the listed candidate mass ions are ranked by the AUC of the ROC curve. The ranking can be from highest to lowest. A predetermined number of candidate biomarker mass ions having the highest ranking are used to generate the multivariate predictive algorithm.

Method 10 for identifying biomarkers and generating an output indicative of lung cancer of the present invention can be used to detect the probable presence of lung cancer in a human subject. A breath sample from a test subject is collected, chemically analyzed and the data is analyzed with the multivariate algorithm to generate a value of the discriminant function for the test subject. The value of the discriminant function for the test subject is compared to the value of the discriminant function determined in block 18.

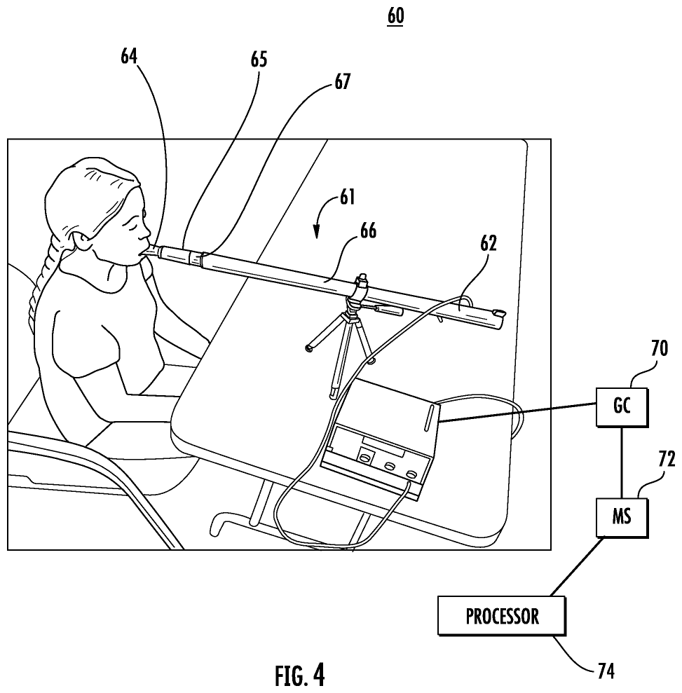

FIG. 4 is a schematic diagram a system for identifying a biomarker to predict nodules visible on a chest CT as indicating lung cancer 60 in accordance with the teachings of the present invention. Breath collection apparatus (BCA) 61 collects samples of volatile organic compounds (VOCs) in alveolar breath and in air onto separate sorbent traps 62. The subject breathes through a disposable valved mouthpiece 64 and a bacterial filter 65. For example, the subject breathes normally for 2.0 min into breath collection apparatus (BCA) 61. Breath reservoir 66 separates alveolar from dead space breath, and alveolar breath is pumped from reservoir 66 through sorbent trap 62. A suitable breath reservoir 66 is a stainless steel tube packed with two grades of activated carbon to capture the VOCs in breath. For example, breath reservoir 66 can capture 1.0 l of breath. A 1.0 l sample of room air is also collected onto second trap 67. A new disposable valved mouthpiece 64 and bacterial filter 65 is employed for every breath collection. For example, each subject can donate two samples for replicate assay at two independent laboratories. An example breath collection apparatus (BCA) is described in U.S. Pat. No. 6,726,637, hereby incorporated by reference into this disclosure.

VOCs are thermally desorbed from the sorbent trap 62, separated by gas chromatography apparatus 70, and injected into mass spectrometry detector 72. In mass spectrometry detector 72 the VOCs are bombarded with energetic electrons in a vacuum and degraded into a set of ionic fragments, each with its own mass/charge (m/z) ratio. Data from gas chromatography apparatus 70 and mass spectrometry detector 72 is received at processor 74.

The unique diagnostic value of the MAGIIC biomarker in this dataset was determined when testing the hypothesis that a single biomarker should predict two conditions simultaneously of pulmonary nodules on a LDCT and biopsy-proven lung cancer. This reduced the universe of 70,000 candidate mass ions biomarkers to a small number of mass ions in which the MAGIIC biomarker delivered the best combination of accuracy, sensitivity and specificity.

Breath tests were performed in a group of 301 subjects undergoing screening for lung cancer. All subjects donated a sample of alveolar breath. Collection of breath VOC samples was performed in accordance with method 10 for identifying biomarkers and generating an output indicative of lung cancer and system 60. A subject wears a nose clip and breathes normally through a disposable valved mouthpiece and bacterial filter into the BCA for 2.0 min. Alveolar breath VOCs are captured on to a sorbent trap that is immediately sealed in a hermetic container. Since there is low resistance to expiration (˜6 cm water), breath samples could be collected without discomfort from elderly patients and those with respiratory disease. In order to minimize the risk of potential site-dependent confounding factors such as environmental contamination of room air, subjects in all four groups donated breath samples in the same room at each clinical site. All subjects donated two samples for replicate assay at two independent laboratories (Menssana Research, Inc referred to as laboratory A and American Westech, Inc., Harrisburg, Pa. referred to as laboratory B). Samples were stored at −15° C. prior to analysis.

Analysis of breath VOC samples: Analysis of breath VOC sample was performed with method 10 for identifying biomarkers and generating an output indicative of lung cancer and system 60. Statistical analysis identified a breath mass ion biomarker mass abnormalities in gaseous ions with imaging correlates (MAGIIC) that correlated with the presence of structural abnormalities in LDCT that were read as consistent with lung cancer. Using automated instrumentation, VOCs were thermally desorbed from the sorbent trap 62, cryogenically concentrated, and assayed by gas chromatography mass spectrometry (GC MS). A known quantity of an internal standard (bromofluorobenzene) was automatically loaded on to all samples in order to normalize the abundance of VOCs and to facilitate alignment of chromatograms. GC MS data from both laboratories was pooled for analysis and development of a single predictive algorithm.

Alignment of single ion masses in chromatograms: Chromatograms were processed with metabolomic analysis software (XCMS in R) in order to generate a table listing retention times with their associated ion masses and intensities. Retention times and ion mass intensities were normalized to the bromofluorobenzene (ion mass 95) internal standard in each chromatogram. The aligned data was then binned into a series of 5 sec retention time segments. Identification of biomarker single ions: The statistical methods have been previously described. Mass ions as candidate biomarkers of lung cancer were ranked by comparing their intensity values in subjects with lung cancer (Group 3 lung cancer confirmed by tissue diagnosis shown in table 3) to cancer-free controls (Group 1 with negative chest CT). In each 5 sec time segment, the diagnostic accuracy of each mass ion was ranked according to its C-statistic value [(area under curve (AUC) of the receiver operating characteristic (ROC) curve]. Multiple Monte Carlo simulations were employed in order to minimize the risk of including random identifiers of disease by selecting the mass ions in each time segment that identified active lung cancer with greater than random accuracy. The average random behavior of mass ions in each time segment was determined by randomly assigning subjects to the “lung cancer” or the “cancer-free” group and performing 40 estimates of the C-statistic. For any given value of the C-statistic, it was then possible to identify the ionic biomarkers that exhibited greater diagnostic accuracy with correct assignment than with multiple random assignments. Development of predictive algorithm: Biomarker ions that identified lung cancer with greater than random accuracy were employed to construct a predictive algorithm using multivariate weighted digital analysis (WDA).

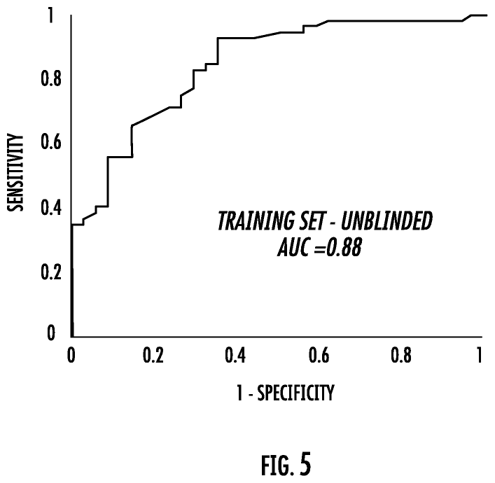

A receiver operating characteristic (ROC) curve of MAGIIC is shown in FIG. 5. It was found that the method of the present invention using the WDA algorithm employing a MAGIIC breath VOC biomarker correlated with nodules observed on a LDCT with sensitivity of 88.0%. FIG. 5 was prepared by constructing ROC curves in Excel spreadsheets and determined sensitivity and specificity employing the following series of steps:

1. List every subject in a row that contains their MAGIIC score and the presence of nodules (yes/no)
2. Use the Excel function to rank all rows according to MAGIIC score, ranging from lowest MAGIIC score in the top row down to highest score in bottom row.
3. Insert two new columns in the spreadsheet labeled sensitivity and specificity
4. For each subject, calculate sensitivity and specificity row by row:

sensitivity=TP/(TP+FN)

specificity=TN/(TN+FP)

where TP=true positives
FN=false negatives
TN=true negatives
FP=false positives
   Accordingly, the top row has sensitivity=100% and specificity=0, and the bottom row has sensitivity=0 and specificity=100%.
   5. Plot the ROC curve with x-axis=(1-specificity), and y-axis=sensitivity.
   6. The point with the optimal combination of sensitivity and specificity corresponds to the point on the ROC curve closest to the top left corner of the graph, where the sum of sensitivity and specificity is maximal.
   7. Plot the sensitivity and specificity on y-axis as a function of MAGIIC score on x-axis as shown in FIG. 5.

The VOC compound names were identified based on mass spectrum of the MAGIIC breath VOC biomarker with known mass spectra of other compounds. In one embodiment, the MAGIIC biomarkers in breath are C4 and C5 alkanes or alkane derivatives. In alternate embodiments, the MAGIIC biomarkers in breath are selected from 1,4-butanediol, 2-pentanamine,4-methyl-, 2-propanamine, 3-butenamide, acetamide, 2-cyano-, alanine, N-methylglycine or octodrine.

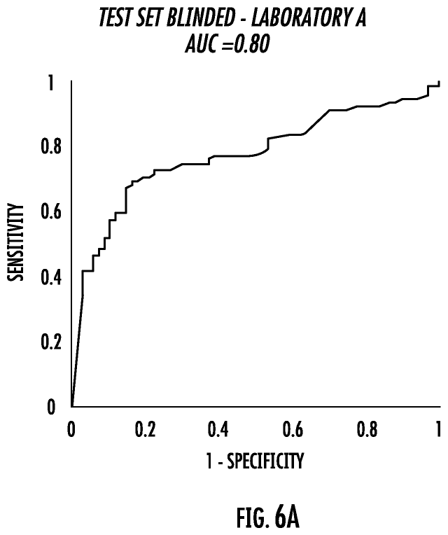

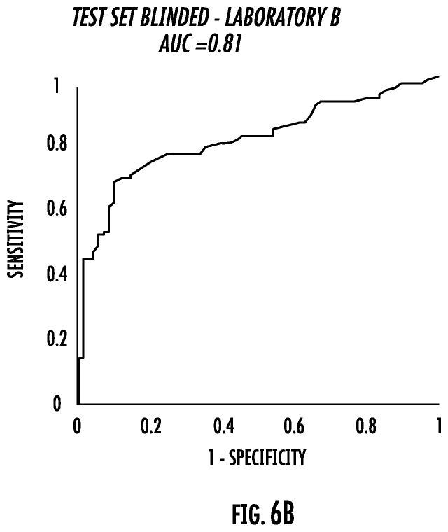

The abundance of the MAGIIC biomarker was determined in a different group of 158 subjects undergoing LDCT. The study was blinded and monitored with Good Clinical Practice (GCP). MAGIIC was assayed in duplicate breath VOC samples analyzed at two independent laboratories, and predicted the outcome of LDCT with 80% at laboratory A as shown in FIG. 6A and predicted the outcome of LDCT with 81% accuracy at laboratory B as shown in FIG. 6B, The present results indicate that ionic biomarkers in breath accurately predicted the presence or absence of nodules in LDCT in a blinded validation study. A multivariate algorithm predicted the diagnosis from replicate breath samples independently analyzed at two laboratories, and the sensitivity, specificity, and overall accuracy of the test were similar at both sites. The outcome of the test was not significantly affected by age or pack-years of tobacco smoking.

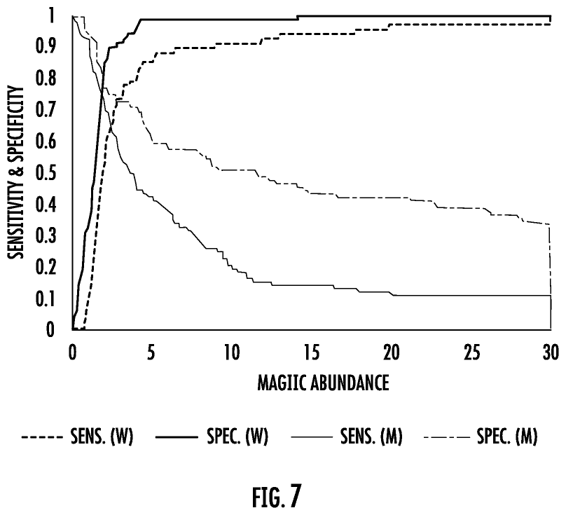

The sensitivity and specificity of MAGIIC for nodules observed on LDCT varied with its abundance in breath is shown in FIG. 7. Abundance of MAGIIC was determined as its ratio to internal standard (bromofluorobenzene) in the chromatogram. The sensitivity at laboratory A is shown as line 101. The specificity at laboratory A is shown as line 102. The sensitivity at laboratory B is shown as line 103. The specificity at laboratory B is shown as line 104. Optimal performance of the test was seen MAGIC abundance=6.4, where the sum of sensitivity plus specificity was maximal and mean sensitivity=80.1% and mean specificity=75.0%. No significant correlation between MAGIIC and smoking could be determined. No correlation was found between the abundance of MAGIIC and pack-years in the set of all current and former smokers. The R\hat{ }2 of pack-years as an ordinary least squares predictor for MAGIIC abundance was approximately 0.01, likely indicative of wide random scatter. It was found that the MAGIIC biomarker was not significantly affected by tobacco smoking.

It is to be understood that the above-described embodiments are illustrative of only a few of the many possible specific embodiments, which can represent applications of the principles of the invention. Numerous and varied other arrangements can be readily devised in accordance with these principles by those skilled in the art without departing from the spirit and scope of the invention.

It is claimed:

1. A method for detecting a biomarker in exhaled breath comprising the steps of:
a. collecting a breath sample from subjects known to have a nodules on a low-dose computed tomography of chest (LDCT) and subjects known to be free of nodules on the LDCT; and
b. analyzing the collected breath samples to identify a subset of mass ions in each of the collected breath samples using at least one time resolved separation technique and at least one mass resolved separation technique to identify one or more target biomarkers in the collected breath samples.

2. The method of claim 1 wherein the subjects are human.

3. The method of claim 1 the subset of the determined mass ions are combined in a multivariate algorithm in a processor to generate a discriminant function and the discriminant function indicates a value of the likelihood that the subject has lung cancer.

4. The method of claim 1 wherein the at least one time resolved separation technique includes gas chromatography and mass spectrometry.

5. The method of claim 1 wherein in step b. of identifying the subset of the determined mass ions further includes the steps of:
classifying the mass ions determined by the at least one time resolved separation technique and at least one mass resolved separation technique mass ions using intensities and retention times;
identifying candidate biomarker mass ions from the classified mass ions;
ranking the candidate biomarker mass ions by diagnostic accuracy for detecting the disease; and
selecting the candidate biomarker mass ions with at least greater than random diagnostic accuracy as the subset of the determined mass ions.

6. The method of claim 5 wherein the step of ranking candidate biomarker mass ions by diagnostic accuracy is determined by the steps of:
determining a receiver operating characteristic (ROC) curve for each of the candidate biomarker mass ions;
evaluating an area under the ROC curve for each of the candidate biomarker mass ions reflecting the diagnostic accuracy for detecting disease;
ranking all candidate biomarker mass ions by the area under the ROC curve for each of the candidate biomarker mass ions;
generating a correct assignment curve with the area under the ROC curve for all of the candidate biomarker mass ions;
generating a random assignment curve with the area under the ROC curve for all of the candidate biomarker mass ions; and
identifying using the correct assignment curve and the random assignment curve the subset of candidate biomarker mass ions with greater than random ability to identify the disease.

7. The method of claim 6 wherein the correct assignment curve and the random assignment curve are generated using Monte Carlo analysis.

8. The method of claim 1, wherein said biomarkers comprise are C4 and C5 alkanes or alkane derivatives.

9. The method of claim 1 further comprising:
a display and further comprising:
controlling the display to display the subset of candidate biomarker mass ions by the processor.

10. A method for detecting the probable presence of lung cancer in a test subject which comprises the steps of:
a. collecting a breath sample from subjects known to have a nodules on a LDCT and subjects known to be free of nodules on a LDCT; and
b. analyzing the collected breath samples to identify a subset of mass ions in each of the collected breath samples using at least one time resolved separation technique and at least one mass resolved separation technique to identify one or more target biomarkers in the collected breath samples.

11. The method of claim 10 wherein the subjects are human.

12. The method of claim 10 the subset of the determined mass ions are combined in a multivariate algorithm in a processor to generate a discriminant function and the discriminant function indicates a value of the likelihood that the subject has lung cancer.

13. The method of claim 10 wherein the at least one time resolved separation technique includes gas chromatography and mass spectrometry.

14. The method of claim 10 wherein in step b. of identifying the subset of the determined mass ions further includes the steps of:
classifying the mass ions determined by the at least one time resolved separation technique and at least one mass resolved separation technique mass ions using intensities and retention times;
identifying candidate biomarker mass ions from the classified mass ions;
ranking the candidate biomarker mass ions by diagnostic accuracy for detecting the disease; and
selecting the candidate biomarker mass ions with at least greater than random diagnostic accuracy as the subset of the determined mass ions.

15. The method of claim 14 wherein the step of ranking candidate biomarker mass ions by diagnostic accuracy is determined by the steps of:
determining a receiver operating characteristic (ROC) curve for each of the candidate biomarker mass ions;
evaluating an area under the ROC curve for each of the candidate biomarker mass ions reflecting the diagnostic accuracy for detecting disease;
ranking all candidate biomarker mass ions by the area under the ROC curve for each of the candidate biomarker mass ions;
generating a correct assignment curve with the area under the ROC curve for all of the candidate biomarker mass ions;
generating a random assignment curve with the area under the ROC curve for all of the candidate biomarker mass ions; and
identifying using the correct assignment curve and the random assignment curve the subset of candidate biomarker mass ions with greater than random ability to identify the disease.

16. The method of claim 15 wherein the correct assignment curve and the random assignment curve are generated using Monte Carlo analysis.

17. The method of claim 10, wherein said biomarkers comprise are C4 and C5 alkanes or alkane derivatives.

18. A system for identifying a biomarker for predicting lung cancer in a subject which comprises:
an apparatus for collecting a breath sample from subjects known to have nodules on a LDCT and subjects known to be free of the disease;
mass spectrometer (MS) associated with a gas chromatograph (GC) apparatus for analyzing the collected breath samples to determine all mass ions in each of the collected breath samples; and
a computer that identifies a subset of the determined mass ions as the biomarkers for detecting lung cancer from the analyzed collected breath samples.

19. The system of claim 18 wherein the subset of the determined mass ions is identified by:
classifying the mass ions determined by the at least one time resolved separation technique and at least one mass resolved separation technique mass ions using intensities and retention times;
identifying candidate biomarker mass ions from the classified mass ions;
ranking the candidate biomarker mass ions by diagnostic accuracy for detecting disease; and
selecting the candidate biomarker mass ions with at least greater than random diagnostic accuracy as the subset of the determined mass ions which are statistically significant for detecting the disease.

20. The system of claim 19 wherein candidate biomarker mass ions are ranked by diagnostic accuracy is determined by:
determining a receiver operating characteristic (ROC) curve for each of the candidate biomarker mass ions;
evaluating an area under the ROC curve for each of the candidate biomarker mass ions reflecting the diagnostic accuracy for detecting the disease;
ranking all candidate biomarker mass ions by the area under the ROC curve for each of the candidate biomarker mass ions;
generating a correct assignment curve with the area under the ROC curve for all of the candidate biomarker mass ions;
generating a random assignment curve with the area under the ROC curve for all of the candidate biomarker mass ions; and
identifying using the correct assignment curve and the random assignment curve the subset of candidate biomarker mass ions with greater than random ability to identify the disease.
